(19) United States
(12) Patent Application Publication
(10) Pub. No.: US 2018/0017776 A1
(43) Pub. Date: Jan. 18, 2018

(54) VARIABLE WORKING DISTANCE MICROSCOPE

(71) Applicant: Novartis AG, Basel (CH)

(72) Inventor: Philip McCulloch, Mansfield, TX (US)

(21) Appl. No.: 15/624,248

(22) Filed: Jun. 15, 2017

Related U.S. Application Data
(60) Provisional application No. 62/361,270, filed on Jul. 12, 2016.

(51) Int. Cl. G02B 21/24; G02B 21/22; H04N 5/225

(52) U.S. Cl. CPC G02B 21/241; H04N 5/2254; A61B 90/20; G02B 21/22

(57) ABSTRACT

A system and method for a variable working distance microscope is disclosed. The variable working distance microscope includes an eyepiece; a binocular system optically coupled to the eyepiece; a stereo zoom system optically coupled to the eyepiece and the binocular system; and a variable working distance lens system optically coupled to the eyepiece, the stereo zoom system, and the binocular system. The variable working distance lens system includes a first lens; a second lens positioned in series with the first lens; and a movable third lens positioned in series between the first and second lenses, the movable third lens configured such that a change in a distance between the moveable third lens and the first lens changes a working distance of a microscope.

### TECHNICAL FIELD

The present invention generally relates to optical microscopes and, in particular, to systems and methods for a variable working distance objective lens microscope.

### BACKGROUND

Optical microscopes are used in a variety of applications to provide the user with an enlarged picture of a specimen in the field of view of the microscope. For example, microscopes may be used in surgical, laboratory, and quality assurance applications. Optical microscopes use visible light and a system of lenses to magnify the specimen.

One type of optical microscope is a common main objective microscope. Common main objective microscopes use a single common main objective lens that is shared between a pair of eyepieces and a lens system.

### SUMMARY OF THE INVENTION

In accordance with some embodiments of the present disclosure, a variable working distance microscope is disclosed. The variable working distance microscope includes an eyepiece; a binocular system optically coupled to the eyepiece; a stereo zoom system optically coupled to the eyepiece and the binocular system; and a variable working distance lens system optically coupled to the eyepiece, the stereo zoom system, and the binocular system. The variable working distance lens system includes a first lens; a second lens positioned in series with the first lens; and a movable third lens positioned in series between the first and second lenses, the movable third lens configured such that a change in a distance between the moveable third lens and the first lens changes a working distance of a microscope.

In accordance with another embodiment of the present disclosure, a variable working distance microscope system is disclosed. The system includes a processor; an image sensor coupled to the processor; a variable working distance lens system optically coupled to the image sensor; and a motor coupled to the processor and the movable third lens and configured to move the third lens to focus an image received by the image sensor. The variable working distance lens system includes a first lens; a second lens positioned in series with the first lens; and a movable third lens positioned in series between the first and second lenses, the movable third lens configured such that a change in a distance between the moveable third lens and the first lens changes a working distance of a microscope.

In accordance with a further embodiment of the present disclosure, a method for focusing a variable working distance microscope is disclosed. The method includes capturing an image at an image sensor of a variable working distance microscope, processing the image by a processor to determine if the image is in focus; and changing a position of a movable third lens until an image at an eyepiece of the variable working distance microscope is in focus. The variable working distance microscope includes a variable working distance lens system. The variable working distance lens system includes a first lens; a second lens positioned in series with the first lens; and a movable third lens positioned in series between the first and second lenses, the movable third lens configured such that a change in a distance between the moveable third lens and the first lens changes a working distance of a microscope.

### BRIEF DESCRIPTION OF THE DRAWING

For a more complete understanding of the present disclosure and its features and advantages, reference is now made to the following description, taken in conjunction with the accompanying drawings, in which:

FIG. 1 is a schematic view of a common main objective microscope including a variable working distance lens system;

FIG. 2 is a schematic view of the variable working distance lens system of FIG. 1;

FIG. 3 is a graph of the working distance of the variable working distance lens system of FIGS. 1 and 2;

FIG. 4 is a graph of the working distance and focal length of the variable working distance lens system shown in FIG. 3;

FIG. 5 is a block diagram of the auto-focus system for a variable working distance lens system shown in FIG. 1; and

FIG. 6 is a flow chart of a method of operating a variable working distance lens system.

### DETAILED DESCRIPTION

The present disclosure provides a common main objective microscope with a variable working distance lens system, allowing an adjustable working distance between the microscope and a specimen, while keeping the overall internal distance within the microscope sufficiently small to allow for the microscope's use while performing activities within arm's reach, such as surgery on the specimen. Due to difficulties in maintaining focus of such a microscope when it is focused manually by a user, the common main objective microscope may also include an auto-focus system.

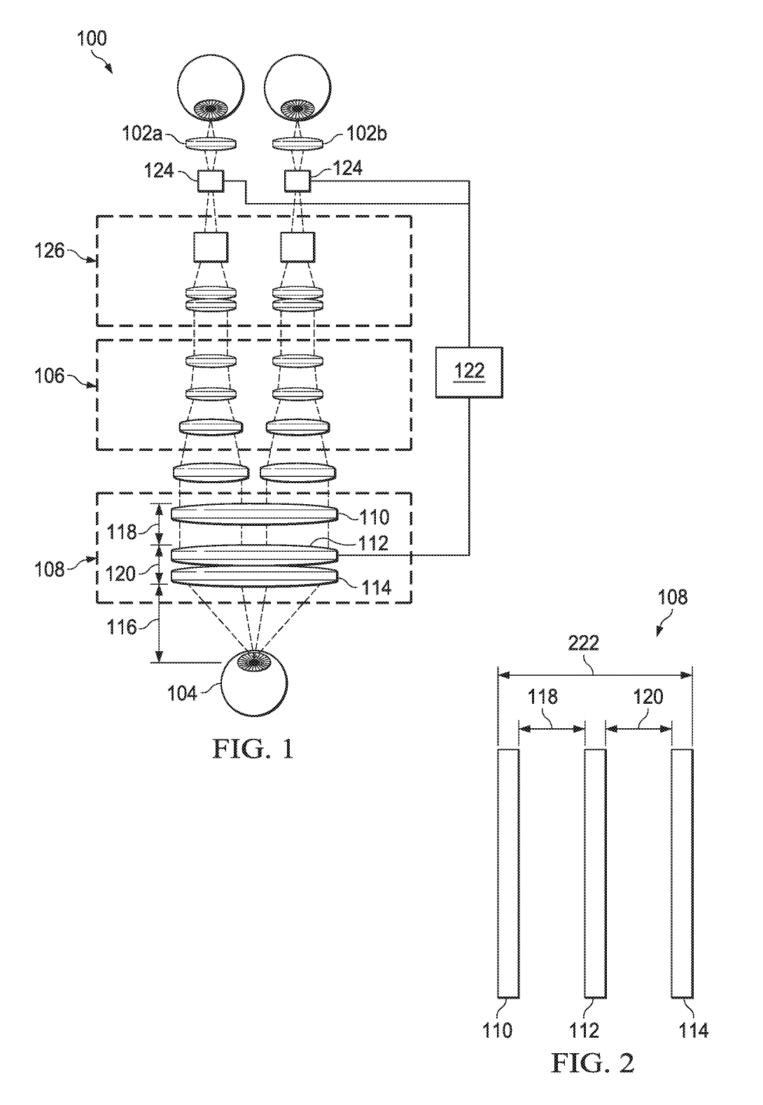

A further description of the embodiments of the common main objective microscope, components thereof, and methods of its uses is presented with reference to FIGS. 1 through 6.

FIG. 1 is a schematic view of a common main objective microscope including a variable working distance lens system. Microscope 100 includes eyepieces 102a and 102b. The user of the microscope views a magnified image of the specimen 104 through eyepieces 102. Specimen 104 is shown in FIG. 1 as an eye, but may be any specimen viewed using microscope 100. Eyepieces 102a and 102b may also be replaced with other components that provide a stereoscopic view of specimen 104, such as two digital displays.

The image of specimen 104 is magnified through the series of stereo zoom lens system 106, variable working distance lens system 108, and binocular system 126. Variable working distance lens system 108 collects light from specimen 104. Stereo zoom lens system 106 may include one or more lenses that may move relative to one another to increase or decrease the magnification of the image of specimen 104 that appears at eyepieces 102. Stereo zoom lens system 106 divides the image of specimen 104 into a stereo view. Binocular system 126 further magnifies the image and provides an image to the user through eyepieces 102.

Variable working distance lens system 108 may replace a common main objective lens and may include lenses 110, 112, and 114 arranged to change working distance 116 of microscope 100. Working distance 116 is the distance between variable working distance lens system 108 and specimen 104. Working distance 116 provides space for the user of microscope 100 to perform actions on specimen 104. For example, where microscope 100 is used for surgery and specimen 104 is the surgical target, working distance 116 is the space available for the surgeon to perform surgical actions using his or her hands and tools. Each user may have a different preference for working distance based on any suitable variable including the size of the user, the type of action to be performed on specimen 104, and the size of the tools used to perform the action on specimen 104.

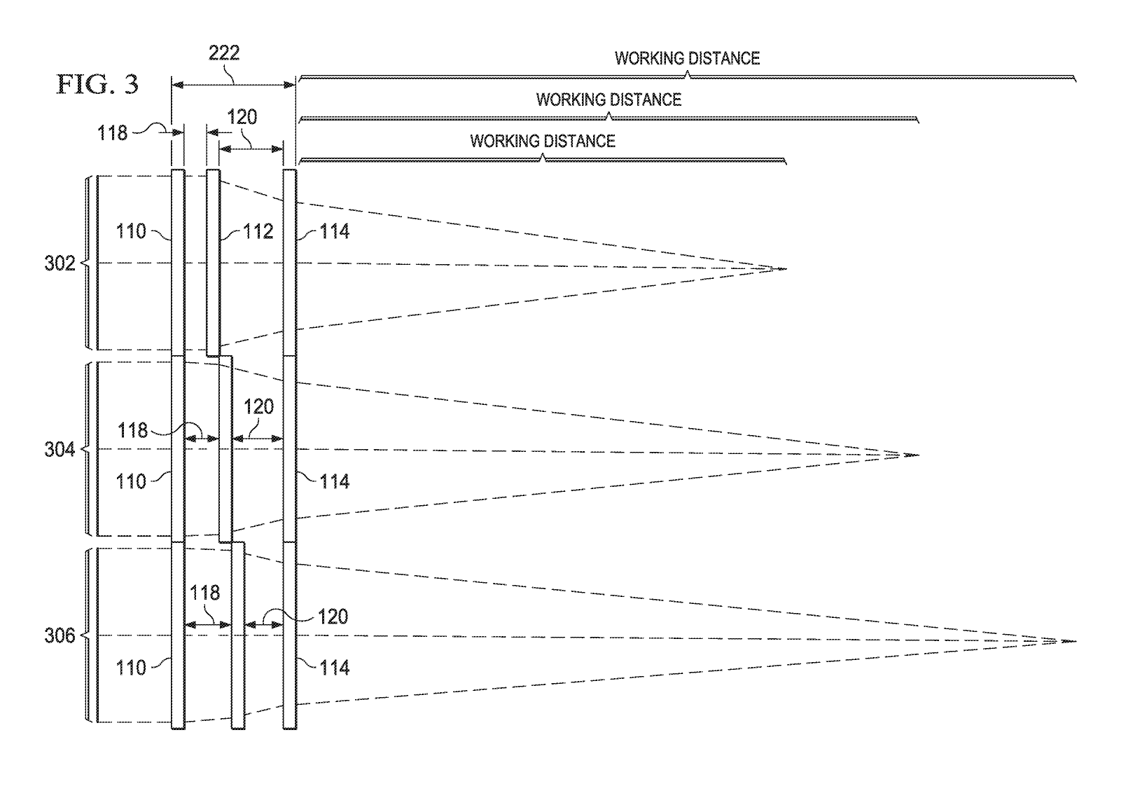

Lenses 110, 112, and 114 may be positioned in series. To change working distance 116, lens 112 within variable working distance lens system 108 may be moved to change distances 118 and 120. Distance 118 is the distance between lens 110 and lens 112. Distance 120 is the distance between lenses 112 and 114. As lens 112 is moved between lens 110 and 114, working distance 116 changes, as explained in further detail with respect to FIGS. 2 and 3. While lenses 110, 112, and 114 are shown as single lenses, each lens 110, 112, and/or 114 may be a single lens or a compound lens. Additionally, while variable working distance lens system 108 is shown in FIG. 1 as including three lenses, variable working distance lens system 108 may include more than three lenses. Furthermore, although only lens 112 moves in the examples discussed herein, systems in which all or more than one lens moves are included in this disclosure.

Microscope 100 may additionally include auto-focus system 122, as shown in more detail in FIG. 5. Auto-focus system 122 may include one or more motors, an image sensor, and a processor. The image sensor may be coupled to beam splitters 124 to allow the image sensor to capture images of specimen 104 as the motor moves lens 112 through the range of positions of lens 112. The processor may execute a software program that determines which of the images captured by the image sensor is in focus. Once the processor determines which image is in focus, the processor may send a command to the motor to move lens 112 to the position at which the in-focus image was captured.

FIG. 2 is a schematic view of the variable working distance lens system of FIG. 1. Variable working distance lens system 200 includes lenses 110, 112, and 114. Lenses 110 and 114 may have fixed positions such that distance 222 between lens 110 and lens 114 does not change. Lens 112 may be movable such that the position of lens 112 may change to change distance 118 between lens 110 and lens 112 and distance 120 between lens 112 and lens 114. Distance 118 and distance 120 have an inverse one-to-one relationship such that when distance 118 decreases by an amount, distance 120 increases by the same amount.

A change in the position of lens 112 changes the focal length and resulting working distance of a microscope including variable working distance lens system 200. The change in the position of lens 112 alters the effective power of lens 110 and has an inverse change on the effective power of lens 114 according to the following equation:

$$\begin{matrix}{φ}_{123} = {φ}_{12} + {φ}_{3} - {φ}_{12} {φ}_{3} ( L - {t}_{1} + {d}_{12}^{′} ) & ( 1 ) \\ where & \\ {φ}_{123} = \frac{1}{{f}_{123}} = the inverse of the focal length of variable working distance lens system 200 ; & \end{matrix}$$

f₁₂₃=the focal length of variable working distance lens system 200;

t₁=distance 118 between lens 110 and lens 112;

t₂=L−t₁=distance 120 between lens 112 and lens 114;

φ₁₂=φ₁+φ₂−φ₁φ₂t₁=the inverse of the focal length of the combination of lens 110 and lens 112;

φ₁=the inverse of the focal length of lens 110;

φ₂=the inverse of the focal length of lens 112; and

φ₃=the inverse of the focal length of lens 114.

A combination of two lenses, such as lens 110 and 112, results in a linear change in the total lens power proportional to the change in the position of lens 112. In order to result in a large change in working distance 116 without variable working distance lens 108 having a size equal to the change in working distance 116, third lens 114 may be added. As such, combining the variables and Equation 1 into a single equation results in the following equation for the combined inverse of the focal length for variable working distance lens system 200:

$${φ}_{123} = ( {φ}_{1} + {φ}_{2} - {φ}_{1} {φ}_{2} {t}_{1} ) + {φ}_{3} - ( {φ}_{1} + {φ}_{2} - {φ}_{1} {φ}_{2} {t}_{1} ) {φ}_{3} ( L - {t}_{1} + \frac{- {φ}_{1} {t}_{1}}{{φ}_{1} + {φ}_{2} - {φ}_{1} {φ}_{2} {t}_{1}} ) \qquad ( 2 )$$

Lenses 110, 112, and 114 may have lens properties and be arranged such that a small change in distance 118 and distance 120 results in a large change in the focal length and working distance of variable working distance lens system 200. For example, an iterative computer program may be used to solve for a combination of lenses 110, 112, and 114 such that the range of working distances for variable working distance lens system 200 is maximized while minimizing distance 222 and thus the overall size of variable working distance lens system 200.

Auto-focus system 122 may use such an iterative computer program. Auto-focus system 122 used with a more complex variable lens system 108 may use modified versions of these equations that take into account additional lenses or the ability of more than one lens to move. An iterative computer program may also be used to solve these modified equations so that the range of working distances is maximized while a distance between one or more lenses is minimized. Auto-focus system 122 is described in more detail with respect to FIG. 5.

FIG. 3 is a graph of the working distance of the variable working distance lens system of FIGS. 1 and 2. The exemplary working distance lens system shown in FIG. 3 includes three lenses. Lens 110 has a focal length of 300 millimeters, lens 112 has a focal length of 115 millimeters, and lens 114 has a focal length of −100 millimeters. The overall size of the variable working distance lens system, distance 222, is 25 millimeters.

FIG. 3 illustrates three positions of lens 112. In position 302, lens 112 is positioned closer to lens 110 than lens 114, such that distance 118 is smaller than distance 120. In position 304, lens 112 is positioned between lens 110 and lens 114, such that distance 118 is approximately equal to distance 120. In position 306, lens 112 is positioned further from lens 110 than lens 114, such that distance 118 is larger than distance 120. As shown in FIG. 3, as lens 112 moves closer to lens 110, the working distance, shown on the x-axis of FIG. 3, decreases. As lens 112 moves closer to lens 114, the working distance increases.

The lenses used in the variable working distance lens system of FIGS. 1-3 are examples that help explain the principles behind the present disclosure. Lenses having other focal lengths may be satisfactorily used in variable working distance lens system 108. Additionally, more than three lenses may be used in variable working distance lens system 108. The lenses may have lens properties and be arranged to result in an approximately 150 millimeter change in the working distance. The lenses may have lens properties and be arranged such that the working distance ranges from approximately 125 millimeters to approximately 275 millimeters. Auto-focus system 122 may be particularly useful in a microscope 100 that contains more total lenses in variable working distance lens system 108 than the three shown in FIGS. 1-3, or in which more than one lens in variable working distance lens system 108 is movable. Such systems may be too difficult for a human to focus.

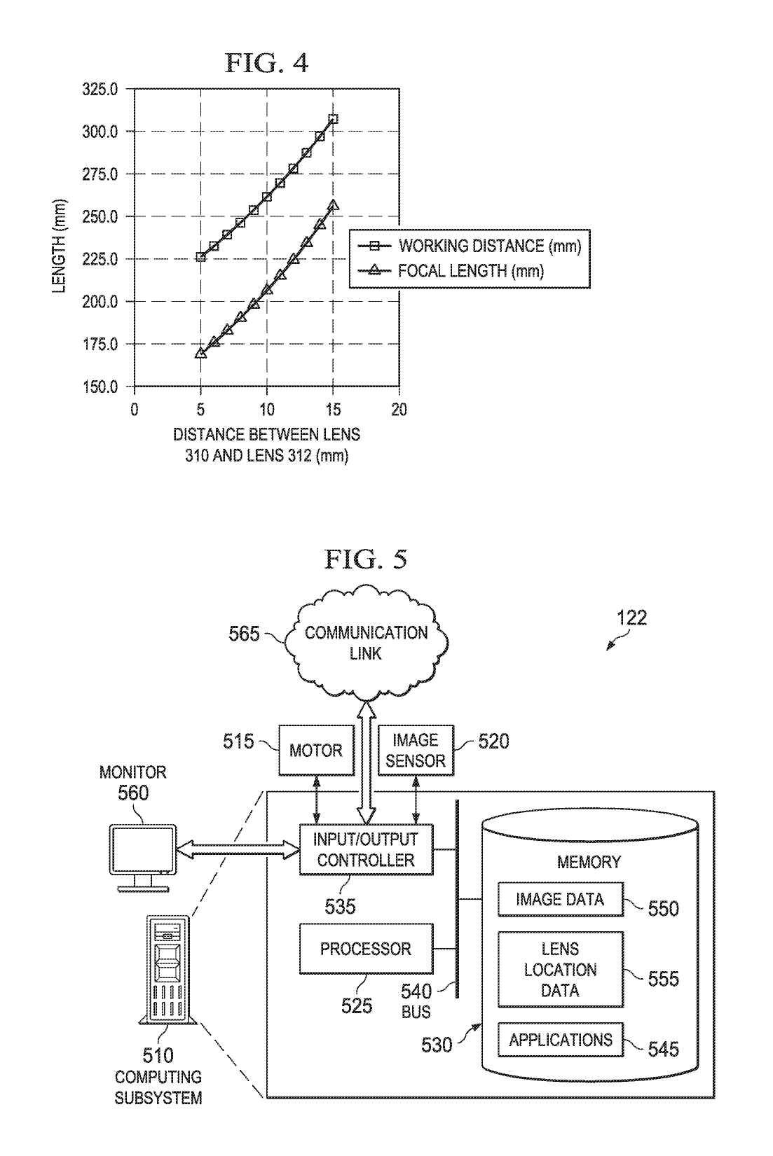

FIG. 4 is a graph of the working distance and focal length of the variable working distance lens system shown in FIG. 3. In the graph, the distance between lens 110 and lens 112 is shown on the x-axis. The distance between lens 110 and lens 112 varies between approximately five millimeters and approximately 15 millimeters. One line represents the working distance for the variable working distance lens system as a function of the distance between lens 110 and lens 112. The working distance ranges from approximately 168 millimeters to approximately 256 millimeters. Another line represents the focal length for the variable working distance lens system as a function of the distance between lens 110 and lens 112. The focal length ranges from approximately 226 millimeters to approximately 307 millimeters. Therefore an approximately 10 millimeter change in the position of lens 112 results in an approximately 90 millimeter change in the working distance and an approximately 80 millimeter change in the focal length. FIG. 4 illustrates how a small change in the distance between lens 110 and lens 112 results in large changes to the working distance and focal length.

FIG. 5 is a block diagram of the auto-focus system for a variable working distance lens system shown in FIG. 1. Auto-focus system 122 may include computing subsystem 510, motors 515, image sensor 520, monitor 560, and communication link 565. Motor 515 may be coupled to a movable lens in a variable working distance lens system, such as lens 112 in FIG. 2. Motor 515 may be activated to change the position of the movable lens and thus change the working distance of the variable working distance lens system. Motor 515 may be any suitable type of motor including a stepper motor, an electric motor, a servomotor, a rotary actuator, a liner actuator, or any combination thereof. The position of the movable lens may be recorded in lens location data 555, discussed in further detail below.

Image sensor 520 may capture images of a specimen in the view field of the microscope, such as specimen 104 shown in FIG. 1. Image sensor 520 may then transmit the images to computing subsystem 510 for storage as image data 550 as discussed in further detail below. Image data 550 may additionally include information related to the position of the movable lens when the image is captured. Image sensor 520 may be any electronic device able to convert light to a digital image. For instance, it may be a digital camera, a light-to-digital sensor, a semiconductor charge-coupled device (CCD), a complementary metal-oxide-semiconductor (CMOS) device, an N-type metal-oxide-semiconductor (NMOS) device, or another electronic device containing an array of photodiodes as part of one or more integrated circuits. Image sensor 520 may contain additional lenses or other elements to assist with image capture. Image sensor 520 produces a digital image with sufficient resolution to produce a usable corrected image, even after image processing.

All or part of computing subsystem 510 may operate as a component of or independent of microscope 100 or independent of any other components shown in FIG. 1. Computing subsystem 510 may include processor 525, memory 530 and input/output controllers 535 communicatively coupled by bus 540. Processor 525 may include hardware for executing instructions, such as those making up a computer program, such as application 545. As an example and not by way of limitation, to execute instructions, processor 525 may retrieve (or fetch) the instructions from an internal register, an internal cache, and/or memory 530; decode and execute them; and then write one or more results to an internal register, an internal cache, and/or memory 530. This disclosure contemplates processor 525 including any suitable number of any suitable internal registers, where appropriate. Where appropriate, processor 525 may include one or more arithmetic logic units (ALUs); be a multi-core processor; or include one or more processors 260. Although this disclosure describes and illustrates a particular processor, this disclosure contemplates any suitable processor.

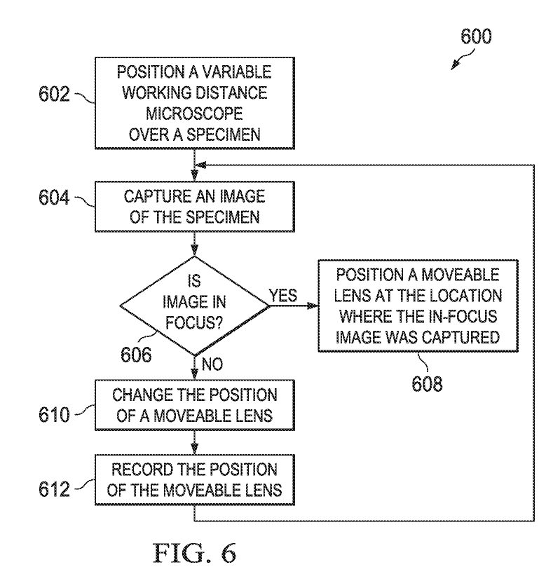

Processor 525 may execute instructions, for example, to auto-focus a variable working distance lens system. For example, processor 525 may run application 545 by executing or interpreting software, scripts, programs, functions, executables, or other modules contained in application 545. Processor 525 may perform one or more operations related to FIG. 6. Input data received by processor 525 or output data generated by processor 525 may include image data 550 and lens location data 555.

Memory 530 may include, for example, random access memory (RAM), a storage device (e.g., a writable read-only memory (ROM) or others), a hard disk, a solid state storage device, or another type of storage medium. Computing subsystem 510 may be preprogrammed or it may be programmed (and reprogrammed) by loading a program from another source (e.g., from a CD-ROM, from another computer device through a data network, or in another manner). Input/output controller 535 may be coupled to input/output devices (e.g., monitor 560, motor 515, image sensor 520, a mouse, a keyboard, or other input/output devices) and to communication link 565. The input/output devices may receive and transmit data in analog or digital form over communication link 565.

Memory 530 may store instructions (e.g., computer code) associated with an operating system, computer applications, and other resources. Memory 530 may also store application data and data objects that may be interpreted by one or more applications or virtual machines running on computing subsystem 510. For example, image data 550, lens location data 555, and applications 545 may be stored in memory 530. In some implementations, a memory of a computing device may include additional or different data, applications, models, or other information.

Image data 550 may include information related to images captured by image sensor 520 that may be used to determine if an image is in focus. Lens location data 555 may include information related to the position of a movable lens in a variable working distance lens system, such as lens 112 shown in FIG. 2. Lens location data 555 may be correlated to each image in image data 550 such that computing subsystem can determine the position of the movable lens for each captured image. Values from image data 550 and lens location data 555 may be used to calculate the position of the movable lens where the image viewed using variable working distance lens system is in focus.

Applications 545 may include software applications, scripts, programs, functions, executables, or other modules that may be interpreted or executed by processor 525. Applications 545 may include machine-readable instructions for performing one or more operations related to FIG. 6. Applications 545 may include machine-readable instructions for calculating when an image viewed through a variable working distance lens system is in focus. For example, applications 545 may be configured to analyze image data 550 to determine when a variable working distance lens system is in focus. Applications 545 may generate output data and store output data in memory 530, in another local medium, or in one or more remote devices (e.g., by sending output data via communication link 565).

Communication link 565 may include any type of communication channel, connector, data communication network, or other link. For example, communication link 565 may include a wireless or a wired network, a Local Area Network (LAN), a Wide Area Network (WAN), a private network, a public network (such as the Internet), a wireless network, a network that includes a satellite link, a serial link, a wireless link (e.g., infrared, radio frequency, or others), a parallel link, or another type of data communication network.

Processor 525 may command motor 515 to move the movable lens, such as lens 112 in FIGS. 1-3, through a range of the positions of the movable lens. While motor 515 is changing the positions of the movable lens, image sensor 520 may record one or more images at various positions of the movable lens. The images may be stored in image data 550 and the lens position corresponding to the image may be stored in lens location data 555. Processor 525 may then execute application 545 to determine which image in image data 550 is in focus. Once application 545 identifies an in-focus image, application 545 may determine the position of the movable lens at which the in-focus image was captured. Processor 525 may then command motor 515 to move the movable lens to the location where the image is in focus.

FIG. 6 is a flow chart of a method of operating a variable working distance lens system. The steps of method 600 may be performed by a person, various computer programs, models or any combination thereof, configured to control and analyze information from microscope systems, apparatuses and devices. The programs and models may include instructions stored on a computer readable medium and operable to perform, when executed, one or more of the steps described below. The computer readable media may include any system, apparatus or device configured to store and retrieve programs or instructions such as a hard disk drive, a compact disc, flash memory or any other suitable device. The programs and models may be configured to direct a processor or other suitable unit to retrieve and execute the instructions from the computer readable media. For example, the programs and models may be one of the applications in applications 545 shown in FIG. 5. For illustrative purposes, method 600 is described with respect to microscope similar to microscope 100 illustrated in FIG. 1; however, method 600 may be used to focus the image of any variable working distance microscope.

Method 600 may begin at step 602 where a user of a variable working distance microscope may position the microscope over a specimen. The user may position the microscope at a position that is ergonomically comfortable for the user to perform a task. For example, a smaller user may position the microscope at a position closer to the specimen than where a larger user may position the microscope.

At step 604, a user or an auto-focus system may capture an image of the specimen. The image may be captured using an image sensor or the image may be captured by the user viewing the specimen through an eyepiece. The auto-focus system may be activated by the user pressing a button on the microscope.

At step 606, the user or an auto-focus system may determine if the image is in focus. A user may determine if the image is in focus by viewing the image and determining if the image appears to be in focus. The auto-focus system may determine if the image is in focus by executing a software application that analyzes the sharpness of the image. If the image is in focus, method 600 may proceed to step 608 where a movable lens is positioned at the location where the image is captured. If the image is not in focus, method 600 may proceed to step 610.

At step 610, the user or an auto-focus system may change the position of the movable lens in a variable working distance lens system. The user may change the position of the movable lens by adjusting a control on the microscope. The auto-focus system may change the position of the movable lens by activating a motor. The motor may be coupled to the movable lens and, when activated, may change the position of the movable lens.

At step 612, the user or an auto-focus system may record the position of the movable lens. The user may record the position by noting the position of the control on the microscope. The auto-focus system may record the position in a database on a computing subsystem. Method 600 may then return to step 604 to capture an image at the current position of the movable lens.

Modifications, additions, or omissions may be made to method 600 without departing from the scope of the present disclosure. For example, the order of the steps may be performed in a different manner than that described and some steps may be performed at the same time. Additionally, each individual step may include additional steps without departing from the scope of the present disclosure.

1. A variable working distance microscope, comprising:
an eyepiece;
a binocular system optically coupled to the eyepiece;
a stereo zoom system optically coupled to the eyepiece and the binocular system; and
a variable working distance lens system optically coupled to the eyepiece, the stereo zoom system, and the binocular system; the variable working distance lens system including:
a first lens;
a second lens positioned in series with the first lens; and
a movable third lens positioned in series between the first and second lenses, the movable third lens configured such that a change in a distance between the moveable third lens and the first lens changes a working distance of a microscope.

2. The variable working distance microscope of claim 1, wherein the moveable third lens includes multiple lenses configured to be moved independently.

3. The variable working distance microscope of claim 1, wherein:
the first lens has a positive focal length;
the second lens has a negative focal length; and
the movable third lens has a positive focal length.

4. The variable working distance microscope of claim 1, wherein the working distance varies by 150 millimeters.

5. The variable working distance microscope of claim 1, wherein the working distance is variable between 125 millimeters and 275 millimeters.

6. The variable working distance microscope of claim 1, wherein at least one of the first lens and the second lens is independently moveable.

7. A variable working distance microscope system, comprising:
a processor;
an image sensor coupled to the processor;
a variable working distance lens system optically coupled to the image sensor, the variable working distance lens system including:
a first lens;
a second lens positioned in series with the first lens; and
a movable third lens positioned in series between the first and second lenses, the movable third lens configured such that a change in a distance between the moveable third lens and the first lens changes a working distance of a microscope; and
a motor coupled to the processor and the movable third lens and configured to move the third lens to focus an image received by the image sensor.

8. The variable working distance microscope system of claim 7, wherein the moveable third lens includes multiple lenses configured to be moved independently.

9. The variable working distance microscope system of claim 7, wherein:
the first lens has a positive focal length;
the second lens has a negative focal length; and
the movable third lens has a positive focal length.

10. The variable working distance microscope system of claim 7, wherein the working distance varies by 150 millimeters.

11. The variable working distance microscope system of claim 7, wherein the working distance is variable between 125 millimeters and 275 millimeters.

12. The variable working distance microscope system of claim 7, wherein at least one of the first lens and the second lens is independently moveable.

13. A method for focusing a variable working distance microscope, comprising:
capturing an image at an image sensor of a variable working distance microscope, the variable working distance microscope including a variable working distance lens system, the variable working distance lens system including:
a first lens;
a second lens positioned in series with the first lens; and
a movable third lens positioned in series between the first and second lenses, the movable third lens configured such that a change in a distance between the moveable third lens and the first lens changes a working distance of a microscope;
processing the image by a processor to determine if the image is in focus; and
changing a position of a movable third lens until an image at an eyepiece of the variable working distance microscope is in focus.

14. The method of claim 13, further comprising:
sweeping a plurality of positions of the movable third lens;
capturing an image at each of the plurality of positions of the movable third lens; and
processing the images to determine which of the images is in focus.

15. The method of claim 14, further comprising:
recording the position of the movable third lens at which each image is captured; and
moving the movable third lens to a position corresponding to the image that is in focus.

16. The method of claim 13, wherein the moveable third lens includes multiple lenses configured to be moved independently.

17. The method of claim 13, wherein:
the first lens has a positive focal length;
the second lens has a negative focal length; and
the movable third lens has a positive focal length.

18. The method of claim 13, wherein the working distance varies by 150 millimeters.

19. The method of claim 13, wherein the working distance is variable between 125 millimeters and 275 millimeters.

20. The method of claim 13, wherein at least one of the first lens and the second lens is independently moveable.
